(19) United States
(12) Patent Application Publication
(10) Pub. No.: US 2001/0049139 A1
(43) Pub. Date: Dec. 6, 2001

(54) Hepatic regeneration from hematopoietic stem cells

(76) Inventors: Eric Lagasse, Palo Alto, CA (US); Irving L. Weissman, Redwood City, CA (US)

Correspondence Address: Pamela J. Sherwood, Bozicevic, Field and Francis LLP, Suite 200, 200 Middlefield Road, Menlo Park, CA, 94025, US

(21) Appl. No.: 09/816,750

(22) Filed: Mar. 23, 2001

Related U.S. Application Data
(60) Provisional application No. 60/191,609, filed on Mar. 23, 2000.

(51) Int. Cl. A01K 67/027; C12N 5/06; C12N 5/08

(52) U.S. Cl. 435/370; 800/18; 435/355

(57) ABSTRACT

Functional hepatic cells are generated from hematopoietic stem cells. In transplantation, populations of hematopoietic stem cells are shown to give rise to repopulating hepatocytes. The stem cells are obtained from a variety of sources, including fetal and adult tissues. The cells are useful in transplantation, for experimental evaluation, and as a source of lineage and cell specific products, including mRNA species useful in identifying genes specifically expressed in these cells, and as targets for the discovery of factors or molecules that can affect them.

The body depends on the liver to perform a number of vital functions, including regulation, synthesis, and secretion of many substances important in maintaining the body's normal state; storage of important nutrients such as glycogen (glucose), vitamins, and minerals; and purification, transformation, and clearance of waste products, drugs, and toxins. However, its distinctive characteristics and activities render it susceptible to damage from a variety of sources, and such damage can have enormous impact on a person's health.

The most abundant and metabolically active cells in the liver are the hepatocytes. The lobules of the liver are hexagonal in shape, with six portal triads at the periphery, each containing a branch of the portal vein, a branch of the hepatic artery, and a bile duct, all held tightly together by a layer of hepatocytes. Hepatocytes rarely divide, but they have a unique capacity to reproduce in response to an appropriate stimulus, such as the removal of a portion of liver. This process involves controlled hyperplasia, that usually restores the liver to within 5 to 10% of its original weight.

Because all hepatocytes can perform the necessary hepatic functions, the liver can undergo compensatory growth and restore its size. Liver regeneration plays an important role after partial hepatectomy and after injuries that destroy portions of the liver, such as viral, toxic, or ischemic damage. However, excessive damage can reach a “point of no return”, and normal tissue is then replaced with scar tissue. The liver's ability to regenerate is also compromised by pre-existing or repeated liver damage or disease.

The existence of hepatic progenitor cells, or stem cells, capable of regenerating both hepatocytes and cholangiocytes, has been debated for many years. Some evidence has supported the existence of such a population, for example see Thorgeirsson (1996) FASEB J. 10:1249-1256. Evidence has indicated that some immature liver cell lines might differentiate into both duct cells and hepatocytes. For example, Fiorino et al. (1998) In Vitro Cell Dev Biol Anim 34(3):247-58 report isolation of a conditionally transformed liver progenitor cell line. Coleman and Presnell (1996) Hepatology 24(6):1542-6 discuss phenotypic transitions in proliferating hepatocyte cultures that suggest bipotent differentiation capacity of mature hepatocytes. Oval cell precursors are thought to be located either in the canals of Herring or next to the bile ducts. Ductal epithelium is required for oval cell proliferation, indicating that either it is the source of the precursors or it acts in a supportive or inductive role.

Hematopoietic stem cells (HSCs) have been rigorously and directly identified. In the BA/Thy1.1 mouse strain, HSCs represent a rare population of 0.01% of whole bone marrow and have been isolated using the combination of cell surface markers: Thy^{lo}Lin^{neg}Scal⁺c-kit^{high}. Descriptions and reviews may be found in Ikuta and Weissman (1992) Proc Natl Acad Sci 89(4):1502-6; Ikuta et al. (1992) Annu Rev Immunol. 10:759-83; Spangrude and Johnson (1990) Proc Natl Acad Sci 87(19):7433-7; and Spangrude et al. (1988) Science 241(4861):58-62.

The functional properties of HSC have been established by transplantation into lethally irradiated host animals under conditions where the progeny of a single stem cell can be identified. These cells are capable of long-term, multi-lineage reconstitution and radioprotection of lethally irradiated host with an enrichment that mirrors their representation in bone marrow by several thousand fold.

Reports have been published indicating that bone marrow cells may be a source of unexpected tissues, such as myocytes, skeletal muscle and most recently hepatocytes. In an example, Bruder et al (1998) Clin Orthop (355 Suppl):S247-56 report that bone marrow contains a population of rare progenitor cells capable of differentiating into bone, cartilage, muscle, tendon, and other connective tissues. These cells, referred to as mesenchymal stem cells, can be purified and expanded in culture from animals and humans. Petersen et al. (1999) Science 284:1168-1170, discloses bone marrow as a potential source of hepatic oval cells. Thiese et al. (2000) Hepatology 31:235-240 discuss the possible derivation of hepatocytes from bone marrow cells in mice after radiation-induced myeloablation.

The further characterization of hepatic progenitor cells is of great scientific and clinical interest.

### RELEVANT LITERATURE

Petersen et al. (1999) Science 284:1168-1170, discloses bone marrow as a potential source of hepatic oval cells. Thiese et al. (2000) Hepatology 31:235-240 discuss the possible derivation of hepatocytes from bone marrow cells in mice after radiation-induced myeloablation.

Mammalian hematopoietic stem cells are described in U.S. Pat. No. 5,087,570, Weissman et al., issued Feb. 11, 1992. Human hematopoietic stem cells are described in U.S. Patent no. 5,061,620, Tsukamoto et al., issued October 29, 1991, herein incorporated by reference.

### SUMMARY OF THE INVENTION

Methods are provided for the generation of functional hepatic cells, which hepatic cells develop from cells having the phenotype of hematopoietic stem cells. Purified populations of hematopoietic stem cells are shown to home to the liver and to give rise to repopulating hepatocytes. The stem cells are obtained from a variety of sources, including fetal and adult tissues. The cells are useful in transplantation, for experimental evaluation, and as a source of lineage and cell specific products, including mRNA species useful in identifying genes specifically expressed in these cells, and as targets for the discovery of factors or molecules that can affect them. In vitro and in vivo systems comprising functional hepatic cells derived from hematopoietic stem cells find use in screening agents that affect hepatocytes, e.g. hepatitis viruses and anti-viral agents, investigating drug metabolism and toxicity, and the like.

### BRIEF DESCRIPTION OF THE DRAWINGS

FIGS. 1A to 1C: isolation of mouse hematopoietic stem cells (HSCs) and expression of CD45 by FACS. A, Phenotypic analysis of bone marrow cells and the different restricted gates used to sort HSCs. B, Analysis of the sorted HSCs (KTLS). HSCs were sorted a second time directly into eppendorf for transplantation. C, CD45 analysis of the sorted HSCs.

FIG. 2: Hematopoietic engraftment 6 months after 1000 HSC transplanted: blood, spleen and bone marrow cells were treated with FDG, and lineage markers Gr-1 for neutrophils, B220 for B cells and Mac-1 and CD3 for myeloid and T cells, respectively. The histograms on the left represent the percentage of donor-derived FDG positive hematopoietic cells. The FACS plots display gates with percentages of donor (right) versus recipient (left) neutrophils and B-cells.

FIGS. 3A to 3B: Separation of bone marrow cells using HSC markers. A, Phenotypic analysis of bone marrow cells from Rosa26/C57BI mice. Density plots of lineage markers, Sca-l and c-kit staining are shown. The percentages in each panel indicate negative or positive cell fractions defined by the gates used for HSC sort. B, Analysis of the sorted bone marrow cells. Lin⁻,Sca-I⁺ and c-kit^{hi} from adult male Rosa26/C57BI mice are separated from Lin⁺, Sca-l⁻ and c-kit⁻, respectively.

FIG. 4 is a schematic illustrating a protocol for engraftment in a non-irradiated host animal.

FIG. 5 is a graph depicting the fluctuation in weight for mice engrafted with hematopoietic stem cells and periodically selected for FAH positive hepatocytes.

### DESCRIPTION OF THE SPECIFIC EMBODIMENTS

A population of cells having the phenotype of mammalian hematopoietic stem cells (HSCs) is demonstrated to have the ability to home to, and regenerate the liver in vivo. The HSCs are useful in transplantation to provide a recipient with restoration of liver function; for drug screening; in vitro and in vivo models of hepatic development; in vitro and in vivo screening assays to define growth and differentiation factors, and to characterize genes involved in liver development and regulation; and the like. The native cells may be used for these purposes, or they may be genetically modified to provide altered capabilities.

The ability to develop HSCs into regenerating hepatocytes can be assessed in vivo in the FAH—/— and FAH knockout immunodeficient animals, e.g. RAG, SCID, nude, etc., with allogeneic, syngeneic or xenogeneic donor cells, by the ability of these donor cells to provide functionality in this system. FAH expression is a defect for the human genetic disorder, tyrosinemia type 1. This function is provided by the engrafted hepatocytes. Alternatively, in vitro methods may be used for the assessment of biological function, by the cultivation of HSC with appropriate growth factors and/or cytokines under hepatocyte generating conditions.

The hematopoietic stem cells are isolated from a source of hematopoietic stem cells, which tissue may be fetal, neonatal, juvenile or adult. The stem cells may be obtained from any mammalian species, e.g. equine, bovine, porcine, canine, feline, rodent, e.g. mice, rats, hamster; primates, including human; etc. The tissue may be frozen and maintained at below about −20° C., usually at about liquid nitrogen temperature (−180° C.).

As used herein, a hematopoietic stem cell (HSC) refers to a primitive or pluripotential hematopoietic stem cell that is capable of giving rise to progeny in all defined hematolymphoid lineages: limiting numbers of stem cells are capable of fully reconstituting lethally irradiated mice, leading to their long-term survival. In humans, the CD34⁺ Thy-1⁺ Lin⁻ hematopoietic stem cells are the equivalent of the murine c-kit⁺ Thy-1.1^{lo} Lin^{-l/o} Sca-1⁺ (KTLS) hematopoietic stem cells and are a virtually pure population of multilineage hematopoietic stem cells. Human HSCs may be further Characterized as AC133 positive; CD38 negative/low; and negative for the specific lineage markers CD2, CD3, CD19, CD16, CD14, CD15, and Glycophorin A. Usually the cell populations used in the present methods are at least about 50% of the cells present having the hematopoietic stem cell phenotype, more usually at least about 75% of the cells present, preferably at least about 85% of the cells present, and may be as high as about 95% of the cells present.

Initial studies have suggested that CD34⁺ bone marrow cells are enriched for pluripotent hematopoietic stem cells (U.S. Pat. No. 5,035,994). U.S. Pat. No. 5,061,620 to Tsukamoto et al. states that B cell and myeloid cell progenitors make up 80-90% of the CD34⁺ cell population. Terstappen et al. (1992) Blood 79:666-677, has suggested that CD34 antigenic density decreases with maturation of hematopoietic cells and increased CD38 cell population. Further studies have shown that CD34 expression is not limited to pluripotent stem cells. When CD34 expression is combined with selection for Thy-1, a composition comprising fewer than 5% of lineage committed cells can be isolated (U.S. Pat. No. 5,061,620). However, recent evidence has suggested that murine hematopoietic stem cells may lack expression of CD34 in the quiescient state (see Goodell et al. (1999) Blood 8:2545-2547).

Methods of determining the presence or absence of a cell surface marker are well known in the art. Typically, labeled antibodies specifically directed to the marker are used to identify the cell population. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids comprising any radionuclides, including, but not limited to, 14 C, 3 H and 35 S. Labeled factors that bind to receptors of interest, e.g. GF-R, are also of interest.

Reagents specific for the human cell surface markers Thy-1 and CD-34 are known in the art and readily available from commercial sources. The murine markers c-kit, Thy-1, and Sca-1 have also been described in the literature and can be detected with readily available reagents.

Lin⁻ refers to cells that are lineage negative, i.e., cells lacking markers such as those associated with T cells (such as CD2, 3, 4 and 8), B cells (such as CD5, CD10, 19 and 20), myeloid cells (such as CD14, 15 and 16), natural killer (“NK”) cells (such as CD2, 16 and 56, NK1.1 for murine cells), RBC (such as glycophorin A, Ter119 for murine cells), megakaryocytes (CD41), mast cells, eosinophils or basophils. Methods of negative selection are known in the art. The absence or low expression of such lineage specific markers may be identified by the lack of binding of antibodies specific to the cell specific markers. Preferably the lineage specific markers include, but are not limited to, at least one of CD2, CD14, CD15, CD16, CD19, CD20, CD38, HLA-DR and CD71; more preferably, at least CD14 and CD15. As used herein, Lin⁻ refers to a cell population selected based on the lack of expression of at least one lineage specific marker. Antibodies specific to lineage specific markers are commercially available from various vendors, e.g. Becton Dickinson, Caltag, AMAC and the ATCC.

Ex vivo and in vitro cell populations useful as a source of stem cells include, but are not limited to, cell populations obtained from bone marrow, both adult and fetal, mobilized peripheral blood (MPB), fetal liver and umbilical cord blood. The use of umbilical cord blood is discussed, for instance, in Issaragrishi et al. (1995) N. Engl. J. Med. 332:367-369. Initially, bone marrow cells can be obtained from a source of bone marrow, including but not limited to, ileum, i.e. from the hip bone via the iliac crest, tibia, femora, vertebrate, or other bone cavities. Other sources of stem cells include, but are not limited to, embryonic yolk sac, fetal liver, and fetal spleen. The methods can include further enrichment or purification procedures or steps for stem cell isolation by positive selection for other stem cell specific markers.

It may be desirable to enrich for the CD34⁺ Thy-1⁺ Lin⁻ cell composition prior to cell transfer or culture. Preferably, the cell population is initially subjected to negative selection techniques to remove those cells that express lineage specific markers and retain those cells which are lineage negative (“Lin⁻”).

Various techniques can be employed to separate the cells by initially removing cells of dedicated lineage. Monoclonal antibodies are particularly useful for identifying markers associated with particular cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. Such separations are up to 10%, usually not more than about 5%, preferably not more than about 1%, of the total cells present not having the marker can remain with the cell population to be retained. The particular technique employed will depend upon efficiency of separation, associated cytotoxicity, ease and speed of performance, and necessity for sophisticated equipment and/or technical skill.

Procedures for separation can include, but are not limited to, physical separation, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of physical separation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). These procedures are well known to those of skill in this art.

Preferred techniques that provide accurate separation include, but are not limited to, flow cytometry, which can have varying degrees of sophistication, e.g., a plurality of color channels, low angle and obtuse light scattering detecting channels, impedance channels, etc. Cells also can be selected by flow cytometry based on light scatter characteristics, where stem cells are selected based on low side scatter and low to medium forward scatter profiles. Cytospin preparations show the enriched stem cells to have a size between mature lymphoid cells and mature granulocytes.

Methods for mobilizing stem cells into the peripheral blood are known in the art and generally involve treatment with chemotherapeutic drugs, cytokines (e.g. GM-CSF, G-CSF or IL3), or combinations thereof. Typically, apheresis for total white cells begins when the total white cell count reaches 500-2000 cells/μl and the platelet count reaches 50,000/μl.

The present methods are useful in the development of an in vitro or in vivo model for hepatocyte functions and are also useful in experimentation on gene therapy and for artificial organ construction. The developing hepatocytes serve as a valuable source of novel growth factors and pharmaceuticals and for the production of viruses or vaccines (e.g., hepatitis viruses), as well as for the study of liver parasites or of parasites having a stage of development in the liver, e.g. malarial organisms), for in vitro toxicity and metabolism testing of drugs and industrial compounds, for gene therapy experimentation (since the liver is the largest vascular organ of the body), for the construction of artificial transplantable livers, and for liver mutagenesis and carcinogenesis studies.

An assay of interest for determining the in vivo capability of hepatic progenitor cells is an animal model of hereditary tyrosinemia type 1, a severe autosomal recessive metabolic disease which affects the liver and kidneys and which is caused by deficiency of fumarylacetoacetate hydrolase (FAH). Treatment of mice homozygous for the FAH gene disruption (FAH^{-/-}) with 2-(2-nitro-4-trifluoro-methylbenzyol)-1,3-cyclohexanedione (NTBC) abolishes neonatal lethality and corrects liver and kidneys functions. The animal model is described, for example, by Grompe et al. (1995) Nature Genetics 10:453-460; Overturf et aL (1996) Nat. Genet. 12(3):266-73; etc.

In one embodiment of the invention, an FAH mouse is reconstituted with human hematopoietic stem cells, in order to provide a chimeric animal useful for screening agents that affect human hepatic cells. The human hematopoietic cells may be introduced into the mouse by any convenient means. For example, the human cells may be introduced into the mouse, which may be an irradiated mouse, and allowed to first reconstitute the bone marrow and other hematopoietic organs, then after reconstitution of hematopoiesis, NTBC is withdrawn in order to select for hepatic reconstitution. Alternatively, NTBC may be withdrawn immediately after introduction of the hematopoietic stem cells. The reconstituted animals are useful for screening vaccines and antiviral agents against hepatic viruses, e.g. Hepatitis A, B, C, D, E; metabolic and toxicity testing of biologically active agents; and the like.

The population of hematopoietic stem cells may also be grown in vitro under various culture conditions, preferably hepatocyte generating culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin.

The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Specific growth factors that may be used in culturing the subject cells include hepatocyte growth factor/scatter factor (HGF), EGF, TGFα, acidic FGF (see JBC vol 132, 1133-1149, 1996), etc. The specific culture conditions are chosen to achieve a particular purpose, ie. maintenance of progenitor cell activity, etc. In addition to, or instead of growth factors, the subject cells may be grown in a co-culture with stromal or feeder layer cells. Feeder layer cells suitable for use in the growth of progenitor cells are known in the art.

The subject co-cultured cells may be used in a variety of ways. For example, the nutrient medium, which is a conditioned medium, may be isolated at various stages and the components analyzed. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions that promote hepatocyte progenitor cell activity.

The stem cell derived hepatocyte progenitors may be used in conjunction with a culture system in the isolation and evaluation of factors associated with the differentiation and maturation of hepatocytes. Thus, the cells may be used in assays to determine the activity of media, such as conditioned media, evaluate fluids for growth factor activity, involvement with formation of specific structures, or the like.

Hepatic failure involves the systemic complications associated with severe liver injury and dysfunction. It may occur in a patient without pre-existing liver disease or may be superimposed on chronic liver injury. The diagnosis of acute liver failure requires the presence of symptoms, including jaundice and encephalopathy. Fulminant hepatic failure impairs all liver functions, causing decreased bilirubin metabolism, decreased clearance of ammonia and gut-derived proteins, and decreased clotting factor production. It may also cause kidney failure, shock, and sepsis. Without a liver transplant, more than 50% of patients will die, usually from a combination of the above conditions. Mortality exceeds 50%, even in the best circumstances. Management involves general supportive measures until the liver can regenerate and resume function. In acute liver failure without pre-existing disease, liver transplant can be life-saving.

The subject cells may be used for reconstitution of liver function in a recipient. Allogeneic cells may be used for stem/progenitor cell isolation and subsequent transplantation. Most of the clinical manifestations of liver dysfunction arise from cell damage and impairment of the normal liver capacities. For example, viral hepatitis causes damage and death of hepatocytes. In this case, manifestations may include increased bleeding, jaundice, and increased levels of circulating hepatocyte enzymes

Liver disease has numerous causes, ranging from microbial infections and neoplasms (tumors) to metabolic and circulatory problems. Hepatitis involves inflammation and damage to the hepatocytes. This type of insult may result from infectious agents, toxins, or immunologic attack. However, the most common cause of hepatitis is viral infection. Three major viruses cause hepatitis in the United States: hepatitis viruses A, B, and C. Together, they infect nearly 500,000 people in the United States every year. In addition, bacteria, fungi, and protozoa can infect the liver, and the liver is almost inevitably involved to some extent in all blood-borne infections.

Numerous medications can damage the liver, ranging from mild, asymptomatic alteration in liver chemistries to hepatic failure and death. Liver toxicity may or may not be dose-related. Tylenol (Acetominophen) is an hepatotoxic drug; Dilantin (an anti-convulsant) and isoniazid (an anti-tuberculosis agent) are examples of drugs that can cause “viral-like” hepatitis. Both environmental and industrial toxins can cause a wide variety of changes in the liver. Hepatic damage is not necessarily dose-dependent and can range from mild, asymptomatic inflammation to fulminant failure or progressive fibrosis and cirrhosis.

Problems with metabolic processes in the liver can be either congenital or acquired. Some of these disorders, such as Wilson's disease and hemochromatosis, can present as hepatitis or cirrhosis. Wilson's disease is a rare inherited condition characterized by an inability to excrete copper into bile, resulting in the toxic accumulation of copper in the liver and nervous system. Hemochromatosis is an iron overload syndrome causing iron deposits and consequent damage to various organs, including the liver, heart, pancreas, and pituitary gland. The disease may be due to an inherited increase in gut absorption of iron or to multiple blood transfusions, since iron is normally found in circulating red blood cells.

The liver may be affected by numerous conditions, particularly autoimmune disorders, in which the immune system attacks the body's own normal tissues. Some examples include rheumatic diseases, such as systemic lupus erythematosus and rheumatoid arthritis, and inflammatory bowel diseases, such as ulcerative colitis and Crohn's disease.

Genes may be introduced into the HSC prior to culture or transplantation for a variety of purposes, e.g. prevent or reduce susceptibility to infection, replace genes having a loss of function mutation, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to transfect the target cells, e.g. electroporation, calcium precipitated DNA, fusion, transfection, lipofection and the like. The particular manner in which the DNA is introduced is not critical to the practice of the invention.

Many vectors useful for transferring exogenous genes into mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For examples of progenitor and stem cell genetic alteration, see Svendsen et al. (1999) Trends Neurosci. 22(8):357-64; Krawetz et al. (1999) Gene 234(1):1-9; Pellegrini et al. Med Biol Eng Comput. 36(6):778-90; and Alison (1998) Curr Opin Cell Biol. 10(6):710-5.

To prove that one has genetically modified progenitor cells, various techniques may be employed. The genome of the cells may be restricted and used with or without amplification. The polymerase chain reaction; gel electrophoresis; restriction analysis; Southern, Northern, and Western blots; sequencing; or the like, may all be employed. The cells may be grown under various conditions to ensure that the cells are capable of differentiation while maintaining the ability to express the introduced DNA. Various tests in vitro and in vivo may be employed to ensure that the pluripotent capability of the cells has been maintained.

The HSCs may be administered in any physiologically acceptable medium, normally intravascularly, including intravenous, e.g. through the hepatic portal vein; intrasplenic, etc. although they may also be introduced into other convenient sites, where the cells may find an appropriate site for regeneration and differentiation. Usually, at least 1×10³/Kg cells will be administered, more usually at least about 1×10⁴/Kg , preferably 1×10⁶/Kg or more. The cells may be introduced by injection, catheter, or the like.

The subject cells are useful for in vitro assays and screening to detect factors that are active on epithelial progenitors. A wide variety of assays may be used for this purpose, including immunoassays for protein binding; determination of cell growth, differentiation and functional activity; production of hormones; and the like.

Of particular interest is the examination of gene expression in stem cell derived hepatocyte and hepatocyte progenitor cells. The expressed set of genes may be compared with a variety of cells of interest, e.g. adult hepatic progenitor cells, stem cells, hematopoietic cells, etc., as known in the art. For example, one could perform experiments to determine the genes that are regulated during development.

Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A⁺ mRNA. One of skill in the art can readily use these methods to determine differences in the size or amount of mRNA transcripts between two samples. For example, the level of particular mRNAs in progenitor cells is compared with the expression of the mRNAs in a reference sample, e.g. differentiated cells.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. Nos. 5,776,683; and 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. Nos. 5,134,854, and 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed at the protein level. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### EXAMPLE 1

### Hepatocyte Regeneration by Hematopoietic Stem Cell Transplantation

Materials and Methods

Mouse Strains:

The mouse strains Rosa26 (C57BIx129sv) (Zambrowicz et al. (1997) Proc Natl Acad Sci USA 94, 3789-94), Rosa26/BA (C57BI/Ka-Thy1.1), FAH−/−(129sv) were bred and maintained in the animal care facility at StemCells.

Staining of HSC:

3 to 6 months old mice were killed to obtain the long bones (two femur and two tibias per mouse). Bone marrow cells were flushed from the long bones with PBS containing 2% fetal calf serum. Cells were stained as described previously (Spangrude et al. (1988) Science 241, 58-62). For KTLS cells isolated from Rosa26 (C57BI/Ka-Thy1.1), the bone marrow cells were incubated with biotinylated mAb specific for Sca-I (Pharmingen), then positively selected using the MACS magnetic bead system (Miltenyl Biotec, Auburn, Calif.). The positively selected cells were stained with phycoerythrin-conjugated lineage markers (Pharmingen), which included the following: RA3-6B2 (B220) for the B lineage marker; RM2-5 (CD2), GK1.5 (CD4), 53-7.3 (CD5), 53.6.7 (CD8) and 145-2C11 (CD3) for T cell markers; RB6-8C5 (GR-1) and M1/70 (CD11b, Mac-1) for myeloid markers; PK136 (NK1.1) for natural killer cells; and Ter119 for erythrocytes. The positively selected cells were also stained with fluorescein-conjugated 19XE5 (Thy1.1), allophycocyanin-conjugated 2B8 (c-kit, Pharmingen) and Streptavidin-Cy7APC (Sav-PharRed, Pharmingen). After the final wash, cells were resuspended in a PBS/FCS buffer that contained propidium iodide (PI, 1 mg/ml) to discriminate between viable and nonviable cells.

Purification of HSC:

Adult bone marrow cell preparations were analyzed by multi-parameter flow cytometry. Isolation of HSC was accomplished using a fluorescence activated cell sorter (FACS™) manufactured by Becton Dickinson Immunocytometry Systems. Specifically, the FACSVantage SE is configured with argon, krypton, and Helium-Neon ion. Computer assisted high speed data acquisition systems allow the collection of up to nine independent data parameters from each single cell. Data parameters were collected in the list mode data file and were analyzed by the software program Flowjo (www.Treestar.com). Pure populations of sorted HSC were resorted directly into eppendorf tubes by an automated cell deposition unit using counter mode. Cells for each group of animal injected were prepared in eppendorfs as follow: 50, 250, 500 and 5 000 HSCs each for a group of 5 mice, respectively. 10⁶ total congenic bone marrow cells from adult FAH−/− female mice were added per eppendorf for a radioprotective dose of 2×10⁵ recipient type bone marrow cells per irradiated FAH−/− mouse. Cells were injected into the retro-orbital plexus of anesthetized mice. One 0.5 ml insulin syringe was used per group of mice to be injected. 100 micro-liters of cells were injected per mouse.

Transplantation Procedure:

The FAH recipient mice is an animal model of hereditary tyrosinemia type 1 (FAH) which has been previously described (Grompe et al. (1995) Nat Genet 10, 453-60). Mice were lethally irradiated with a total dose of 1200 rads in a split dose with 3 hours interval. One day later, cells were injected intravenously into the retro-orbital plexus of anesthetized mice using insulin syringes (Becton Dickinson, Franklin Lakes, N.J.). All the experimental FAH mice were treated with 2(2-nitro-4-trifluoromethylbenzoyl)-1,3 cyclohexane dione (NTBC) containing drinking water before and for the next 2 months after the irradiation procedure. To evaluate the level of reconstitution, peripheral blood was collected 2 months after the transplantation and samples were monitored for donor-marked cells (Rosa26 beta-galactosidase positive cells) and for specific lineage markers (B220 for B cells, CD3 for T cells and Mac-1 and GR-1 for myeloid cells). Two weeks after the bleeding, NTBC was discontinued to permit positive selection of hepatocytes to occur in the liver. The weight of experimental animals was monitored weekly and NTBC added back to the water when the mouse weight was reaching under 20 grams for an adult mouse.

Histology and Immunohistology:

Liver was embedded in OCT and frozen in liquid nitrogen. Serial sections of 5 and 10 micron-thick were stained histochemically for beta-galactosidase and immunohistochemically with the polyclonal rabbit anti-FAH antibody. For some samples, the median lobe of the liver was fixed in 4% paraformaldehyde at 4° C. overnight and stained for beta-galactosidase.

Detection of Beta-galactosidase:

Fluorescein di-beta-D-galactopyranoside (FDG, Molecular Probe) was used as the fluorogenic substrate to detect beta-galactosidase by flow cytometry. 5-bromo-4-chloro-3-indolyl-beta-D-galactopyranoside (X-Gal) was used as the substrate to detect beta-galactosidase in sections.

Fluorescent In Situ Hybridization (FISH):

For performing FISH on cryostat sections, 5 μm sections of the targeted tissue are prepared and stored unfixed at −80° C. When ready to begin the FISH procedure, the sections are thawed to room temperature, fixed 3 times in Carnoy's Fixative for 10 minutes each and allowed to air dry at room temperature. The sections are then pre-treated at 37° C. for 30 minutes in preheated 2×SSC Buffer pH 7.0. After pretreatment, serial ethanol dehydration (70%-70%-90%-90%-100%) is done for 1.5 minutes each and again the slides air-dry at room temperature. Specific denaturing conditions are established for each type of probe and are important to ensure proper hybridization. In this case, the slides are denatured in preheated 70% Formamide/2×SSC Buffer pH 7.0 at 65° C. for 2 minutes. The slides are then immediately quenched with ice cold 70% ethanol for 1.5 minutes. Serial ethanol dehydration is done again as described above and the slides are air-dried. The Cambio STAR*FISH Mouse-Y chromosome FITC labeled probe is prepared ahead of time by thawing the tube to 37° C. The appropriate aliquot removed is then denatured at 65° C. for 10 minutes and kept at 37° C. until ready to apply to the slides. Again, as with the denaturing conditions, the preparation of the probe is specific for each probe type. The prepared probe is applied to the air-dried slides while on the slide warmer set at 45° C. The slides are coverslipped and sealed with rubber cement for incubation overnight in a hydrated slide box at 42° C. The following day, the coverslips are carefully removed in preheated 2×SSC Buffer pH 7.0 at 45° C. The slides are then stringently washed twice in preheated 50% Formamide/2×SSC Buffer for 5 minutes each at 45° C. and then gently washed twice in preheated 0.1×SSC Buffer for 5 minutes each at 45° C. The appropriate detection and/or counterstain protocols, included with each probe, should be followed to view the hybridization under a fluorescent microscope. In this case, a directly labeled probe is used and only counterstaining with Hoechst and/or Propidium iodide is necessary. For performing FISH on cell drop preparation, slides were treated following the Cambio protocol.

The experimental data provided herein addresses whether bone marrow-derived cells are heterogenous in nature and contain several type of stem cells or progenitors cells for different tissues or alternatively whether bone marrow-derived cells may be homogenous in nature with HSCs retaining the capacity to differentiate into other tissue types under the appropriate conditions. Further, it is determined whether HSC provide for liver repopulation in the form of regenerative hepatic nodules, which is the hallmark of functional hepatocytes repairing damaged or diseased liver and would have major implications for the use these cells for gene and/or cell therapy.

It was tested if highly purified HSCs could give rise to hepatocytes in the FAH−/− mouse, an animal model of hereditary tyrosinemia type 1. These mice suffered from a severe autosomal recessive metabolic disease which affects the liver and kidneys and which is caused by deficiency of fumarylacetoacetate hydrolase (FAH). Treatment of mice homozygous for the FAH gene disruption (FAH−/−) with 2-(2-nitro-4-trifluoro-methylbenzyol)-1,3-cyclohexanedione (NTBC) abolished neonatal lethality and correct liver and kidneys functions. We used FAH−/− mice as recipient for the engraftment of HSCs because this model allows a strong growth advantage of wild-type hepatocytes to repopulate mutant liver (Overturf, et al. (1996) Nat Genet 12, 266-73).

Result and Discussion

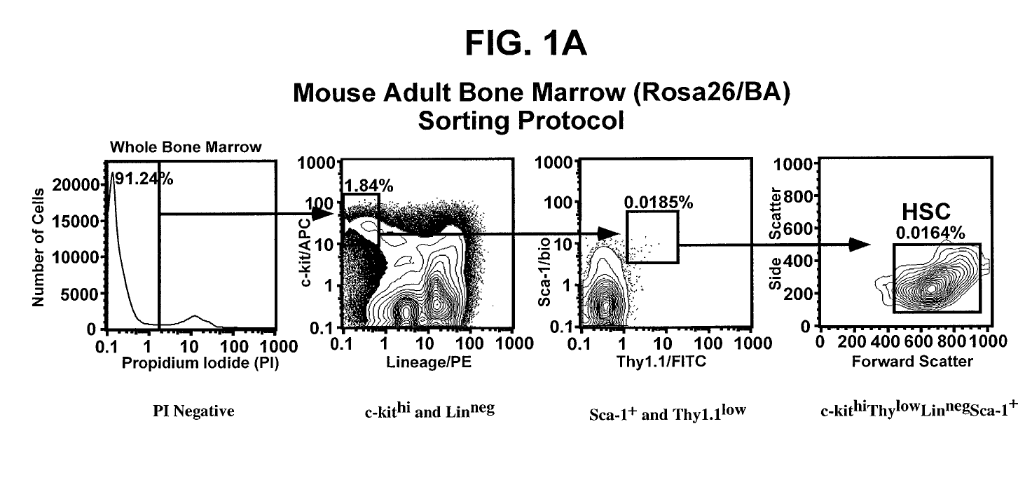

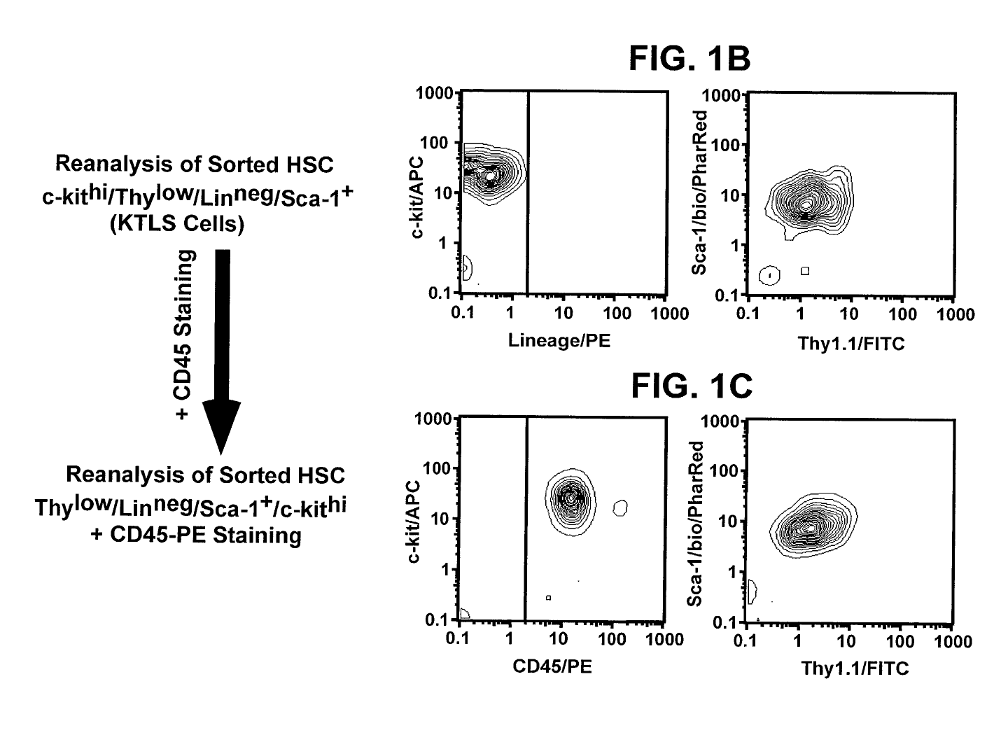

HSCs were isolated from the bone marrow of normal adult male Rosa26/BA mice by fluorescence-activated cell sorting (FACS) (FIG. 1). These HSCs, also termed KTLS for the markers c-kit^{high}Thy^{lo}Lin^{neg}Scal³⁰ , are c-kit high, Thy1.1 low, lineage marker (CD2, CD3, CD4, CD5, CD8, NK1.1, B220, Ter119, GR-1 and Mac-1) negative to low, and Sca-1 positive. Sorted HSCs were stained for CD45, the leukocyte common antigen (LCA) also known as LY-5 or T200, found on all cells of hematopoietic origin, except erythrocytes. Its presence distinguishes leukocytes from non-hematopoietic cells. CD45 was detected on all sorted KTLS cells from Rosa26/BA mice indicating that the HSCs population isolated is hematopoietic in its origin.

10, 50, 100 or 1000 (KTLS) HSCs were injected intravenously into lethally irradiated adult female FAH−/− mice with 2×10⁵ FAH−/− congenic adult female bone marrow cells as a radioprotective dose. NTBC was kept in the drinking water for the first 2 months of the experiment because it was known from previous experiments that lethally irradiated FAH−/− mice will die rapidly of acute liver failure if NTBC is withdrawn just after irradiation. Two months after HSCs transplantation, nucleated blood cells of the experimental animals were tested for hematopoietic engraftment. As shown in Table 1, most of the animals were engrafted 2 months after being transplanted with 10 HSCs to 1000 HSCs and the number of HSCs injected was proportional to the corresponding amount of reconstituted blood cells.

TABLE 1
Analysis of blood cells 2 Months after Transplantation

| | | | % of Rosa26 | |
|---|---|---|---|---|
| | | | Positive Cells | |
| Number | | | Total | |
| of cells injected | | Survival | Blood | Neutrophils |
| 10 HSC from Rosa 26 | F#597 | Dead 8/17/99 | | |
| male mice + 2 × 10⁵ | F#598 | | 1.04 | 0.44 |
| Bone Marrow from | F#599 | | 2.01 | 0.96 |
| FAH −/− Female Mice | F#600 | | 0.51* | 0.13* |
| | F#601 | | 0.18* | 0.03* |
| 50 HSC from Rosa 26 | F#602 | | 6.80 | 6.14 |
| male mice + 2 × 10⁵ | F#603 | | 2.85 | 6.23 |
| bone marrow from | F#604 | | 0.23* | 0.20* |
| FAH −/− female mice | F#605 | | 3.10 | 3.87 |
| | F#606 | | 3.27 | 2.98 |
| 100 HSC from Rosa | F#607 | | 0.89 | 0.66 |
| 26 male mice + 2 × | F#608 | | 10.33 | 16.23 |
| 10⁵ bone marrow from | F#609 | | 7.66 | 2.85 |
| FAH −/− female mice | F#610 | | 16.11 | 1.06 |
| | F#611 | | 16.58 | 4.05 |
| 1000 HSC from Rosa | F#612 | Dead 8/23/99 | | |
| 26 male mice + 2 × | F#613 | Dead 8/23/99 | | |
| 10⁵ bone marrow from | F#614 | | 58.76 | 73.59 |
| FAH −/− female mice | F#615 | | 32.71 | 42.26 |
| | F#616 | | 42.15 | 39.34 |
| Hematopoietic donor-derived cells were detected in blood, spleen and bone marrow by FACS using the FDG fluorogenic substrate. Numbers are presented as % of nucleated donor-derived cells found in the tissue and correspond to an average of 2 samples. 3 mice died in the first 2 months and were not analyzed. <1 indicated no detectable engraftment. B, T, M stand for B cells, T cells and Myeloid cells and were identify by B220, CD3 and | | | | |
| # GR-1 + Mac-1 antibodies in combination with FDG staining. | | | | |

*These mice died after their cage flooded. For liver engraftment, + stands for the identification of donor-derived hepatocytes.

TABLE 2
Detection of donor-derived cells 6 months after transplant

| | | | |
|---|---|---|---|
| | Number of | | |
| | Cells | | |
| | Transplanted + | | |
| Donor | 2 × 10⁵ | Hematopoiesis | Liver |

| Cell Type | FAH^{-/-} BM | Blood | Spleen | BM | Nodules | Hepatocytes |
|---|---|---|---|---|---|---|
| c-kit + | 20,000 | 13.3 B,T,M | 52.0 B,T,M | 44.7 | 35 | + |
| c-kit − | 135,000 | <1 | <1 | <1 | 2* | − |
| Lin + | 205,000 | 1.5 B,T | 2.9 B | 2.6 | 7* | − |
| Lin − | 20,000 | 33.7 B,T,M | 56.5 B,T,M | 34.0 | >70 | + |
| Sca-1 + | 23,000 | 28.7 B,T,M | 13.8 B,T,M | 75.4 | 55 | + |
| Sca-1 − | 224,000 | 3.5 B,T,M | 8.2 B,T,M | 3.5 | 12 | + |
| Detection of donor-derived cells 6 months after transplantation. Numbers are presented as % of nucleated donor-derived cells and correspond to an average of 2 samples. <1 correspond to no detectable engraftment. B, T, M stand for B cells, T cells and Myeloid cells and were identify by B220, CD3 and GR-1 + Mac-1 antibodies in combination with FDG staining. For liver, 25 serial sections per donor cell type were scanned and the numbers represent X gal positive hepatocytes | | | | | | |
| # counterstained with Hoeschst. | | | | | | |

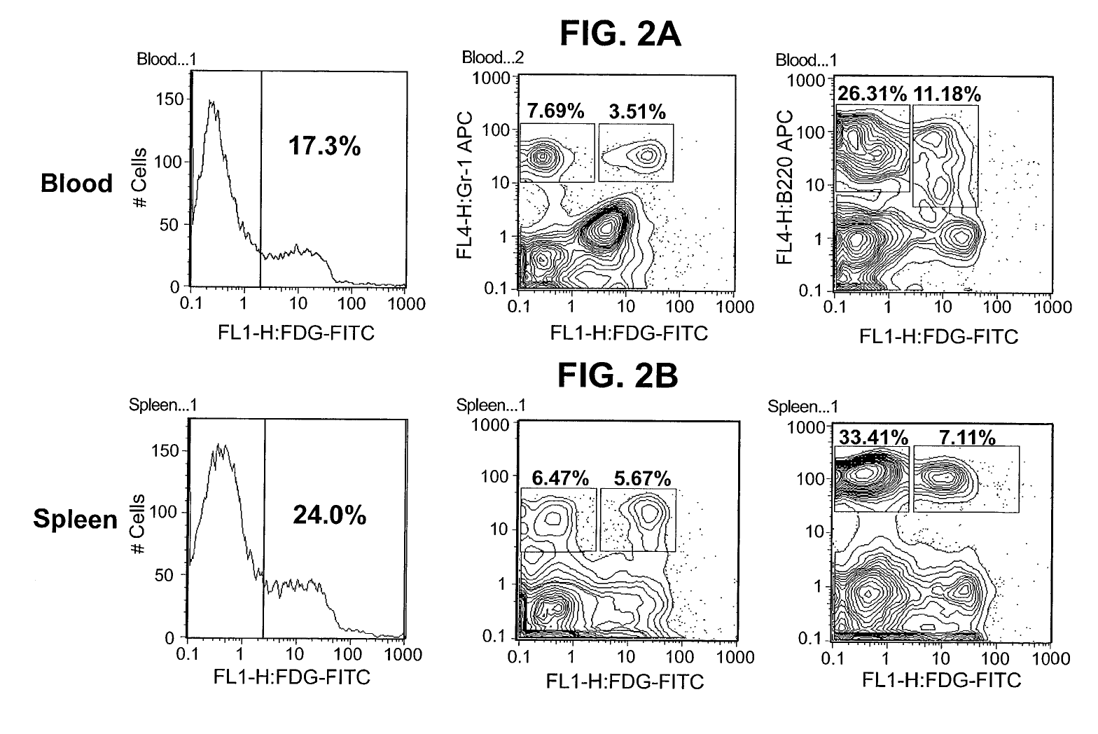

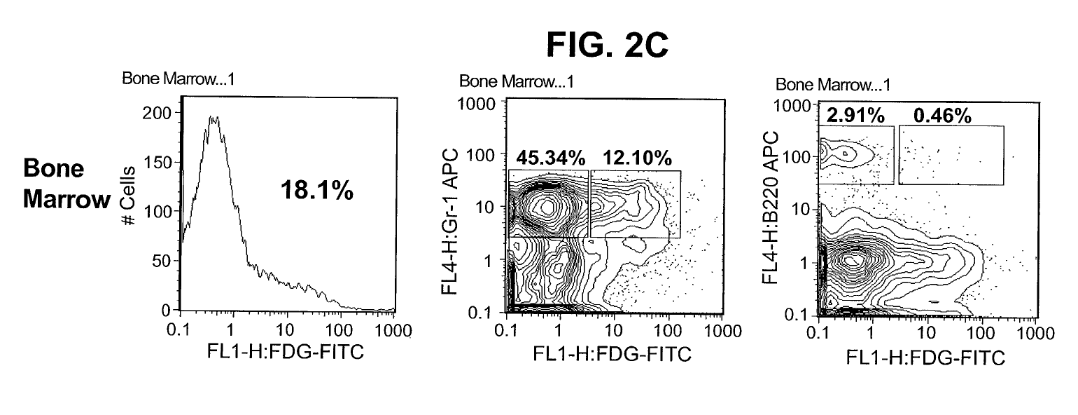

During the next 4 months, positive selection was applied twice to the FAH−/− mice by removing NTBC from the drinking water. NTBC was added back to all experimental animals if the weight of a mouse fell too low. Mice surviving the treatment were sacrificed after the second selection (6 months post HSC transplant). Bone marrow, blood and spleen were analyzed as single cell suspension by FACS for multilineage reconstitution (B, T and myeloid lineages) of the hematopoietic system (Table 1 and FIG. 2). This analysis confirm that the hematopoietic system from all the surviving host FAH−/− mice were engrafted long term with male Rosa26 HSCs. It is interesting to note that we have engrafted HSCs across minor histocompatibility barriers.

For hepatic engraftment, the degree of repopulation achieved was monitored by several criteria. The whole median lobe of the liver of most experimental animals was fixed and stained to detect any macroscopic nodules. For the rest of the liver, serial sections were analyzed for donor-derived hepatocytes by the following criteria: the presence of beta-galactosidase positive cells by histochemical staining, the expression of FAH protein within the hepatocytes by immunostaining and the appearance of male donor cells by fluorescent In Situ Hybridization (FISH) of Y chromosome. Nodules of X-gal positive activity were detected in liver of mice injected from 50 to 1000 HSCs. Nodules were small and discrete from 50 hepatocytes to large with over 10⁵ hepatocytes. Liver histology demonstrate X-gal positive hepatocytes in the nodules analyzed. Frozen section analysis of serial sections shows a co-expression of FAH with beta-galactosidase in the repopulating hepatocytes. Furthermore, these nodules were also shown to contain Y-chromosome positive nuclei. The finding that hepatocytes are X-gal, positive, co-expressed FAH protein and are Y chromosome positive indicate that they are derived from the donor HSCs. In addition, the clustering and regional replacement of the diseased parenchyma by HSC-derived hepatocytes demonstrates the potential role HSCs could have in cell therapy of the liver.

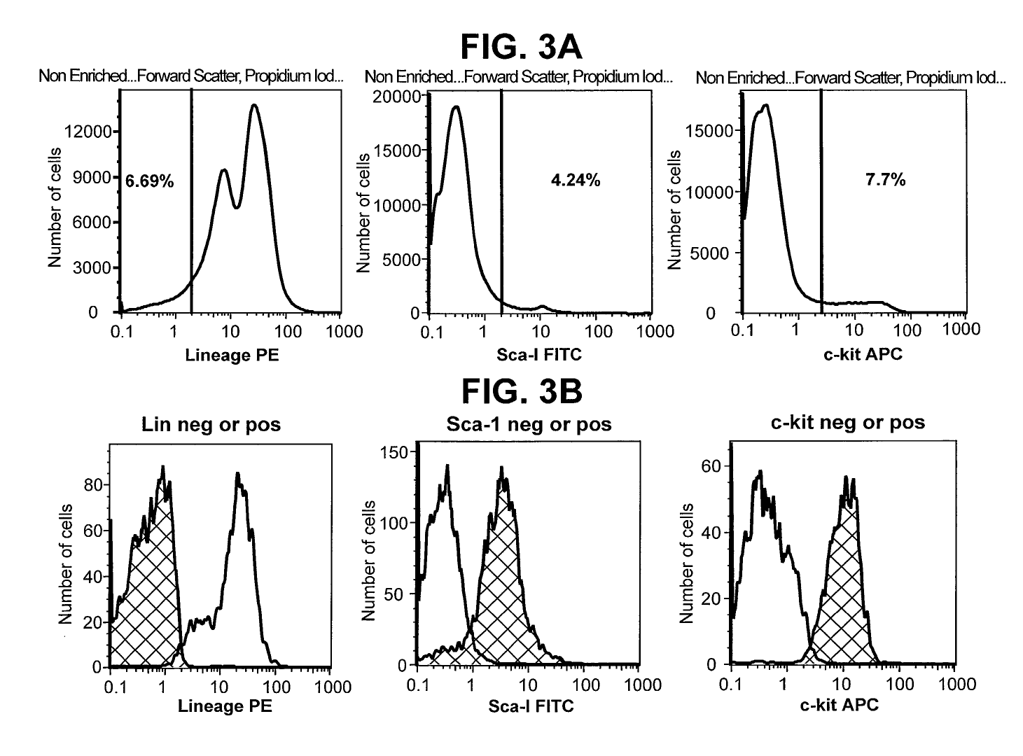

In a second set of experiment, it was tested whether HSC markers c-kit^{high} or Lin^{neg} or Scal⁺ cells are the only cells in the bone marrow that contain the hepatic progenitors. To avoid excluding any cell populations, bone marrow was divided among c-kit+ versus c-kit− pools, Lin+ versus Lin− pools and Scal+ versus Scal− pools using flow cytometry (FIG. 3). If hepatic progenitors were expressing these antigens uniformly, hepatic engraftment would be enriched in one fraction and correspondingly depleted in the other. As for the previous experiment with HSC, Rosa26 bone marrow subpopulations were injected intravenously into lethally irradiated FAH−/− mice along with 2×10⁵ FAH−/− congenic adult female bone marrow as a radioprotective dose. One month later NTBC was removed from the drinking water and twice during the 4 next months, positive selection was applied similarly to the above experiment with HSCs. Only one of the mice for each group survived the positive selection. Mice were sacrificed and hematopoietic and hepatic engraftment evaluated. For hematopoiesis, blood, spleen and bone marrow cells were analyzed for donor cells (FIG. 3). For the liver engraftment, 25 serial sections of 10 micron each were analyzed and X-gal positive donor hepatocytes counted. c-kit− cells (representing 92.3% of WBM), Lin+ cells (representing 93.4% of WBM) and Sca-l− (representing 95.8% of WBM) did not contribute significantly to long-term multi-lineage reconstitution in a previous reported study and did not provide an enrichment in hepatocyte engraftment. Long-term multi-lineage reconstitution and hepatocyte engraftment was the property of markers c-kit+ (7.7% of WBM), Lin− (6.6% of WBM) and Sca-l+ (4.2% of WBM) cells. c-kit^{neg}, Lin^{pos}, Scal⁻ cells represents 99.9% of the bone marrow and do not possess stem cell activity (hematopoietic or hepatic). Only when HSCs engrafted with long-term multi-lineage reconstitution was hepatocyte engraftment seen.

The data demonstrate that the same HSCs which give rise to the hematopoietic system in these mice also have the plasticity to give rise to hepatocytes. It is shown that bone marrow cells can rescue a metabolic disorder of the liver by regenerating hepatocytes. As few as 50 HSCs can engraft both the hematopoietic and the hepatic compartments. Finally it is shown that only the HSCs fraction of the bone marrow have the plasticity to give rise to hepatocytes.

### EXAMPLE 2

### Enhanced Biopotency for Differentiation of HSC into Hepatocytes with Mobilized Adult Blood

Treatment with a wide variety of chemotherapeutics or cytokines leads to an increase in the frequency of hematopoietic progenitor cells in the peripheral blood. Cyclophosphamide (CY) and granulocyte colony-stimulating factor (G-CSF) treatment of mice increases the fraction of bone marrow HSC in S-phase of the cell cycle, leading to an expansion of the number of bone marrow HSC prior to mobilization into the peripheral blood. Mobilized HSC tended to be in G0/G1 phase, are less efficient than normal bone marrow multipotent progenitors in hematopoietic engraftment of irradiated mice, but do not differ in colony forming unit-spleen (CFU-S) activity or single cell in vitro assays of primitive progenitor activity. KTLS HSC (using the markers as described in Example 1) were isolated from ROSA26/BA mice treated with CY and G-CSF by flow cytometry after Sca-1 enrichment using a MACS column (Miltenyi Biotec). 700 sorted HSC were injected intravenously into lethally irradiated adult female FAH⁻/⁻ mice with (10 mice) and without (10 mice) 2×10⁵ FAH⁻/⁻ congenic adult bone marrow cells. NTBC was kept in the drinking water for the first 2 months of the experiment.

Two months after mobilized KTLS HSC transplantation, nucleated blood cells of the experimental animals were tested for hematopoietic engraftment as described above. All the animals were engrafted at 2 months with 700 mobilized KTLS HSC. The animals injected with only 700 HSC were over 90% engrafted while the animal injected with 700 mobilized KTLS HSC and congenic bone marrow had a lower level of engraftment. Positive selection was applied once to the FAH^{-/-} mutant liver by removing NTBC from the drinking water and restarting the drug when total body weight decreased by more than 30%. Several mice were sacrificed after this first selection (3 months after HSC transplantation). Bone marrow, blood and spleen were analyzed as single cell suspensions by FACS for donor-specific multilineage reconstitution (B, T and myeloid lineages) of the hematopoietic system. This analysis confirmed that the hematopoietic systems of all the analyzed host FAH^{-/-} mice were engrafted long-term with donor male ROSA26 HSC. The degree of hepatic engraftment achieved was monitored by several methods. Serial sections were analyzed for donor-derived hepatocytes by the following criteria: the presence of beta-galactosidase positive cells by histochemical staining and the expression of FAH enzyme within the hepatocytes by immunostaining. It was found that HSC-derived hepatocytes were present in most animals analyzed. Interestingly, hepatic engraftment was much more rapid than in previous experiments using adult bone marrow HSC. With adult bone marrow HSC, HSC-derived hepatocytes could only be scored in sections 6 months after transplantation. Strikingly, hepatic engraftment was evident 3 months after transplantation of mobilized KTLS HSC.

Methods

Briefly, mice (ROSA26/BA) were injected i.p. with 4mg of CY (˜200 mg/kg) and then on four successive days with 5 micrograms of human G-CSF (˜250 microg/kg per day) administered as a single daily s.c. injection. Mouse blood was collected 1 day after the last G-CSF injection and mobilized HSC were isolated by MACs selection (Sca1 positive selection) and cell sorting.

### EXAMPLE 3

### HSC are “Natural” Progenitors for Hepatocytes

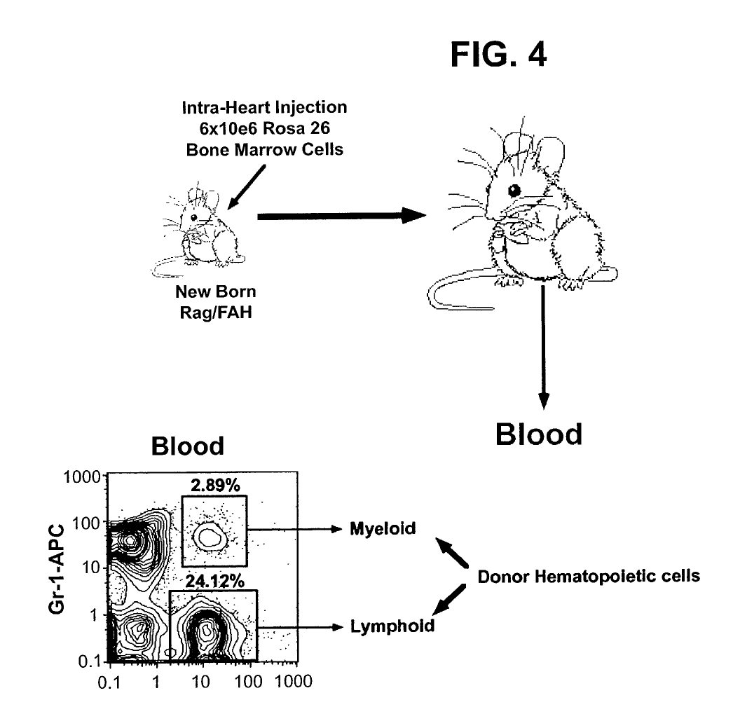

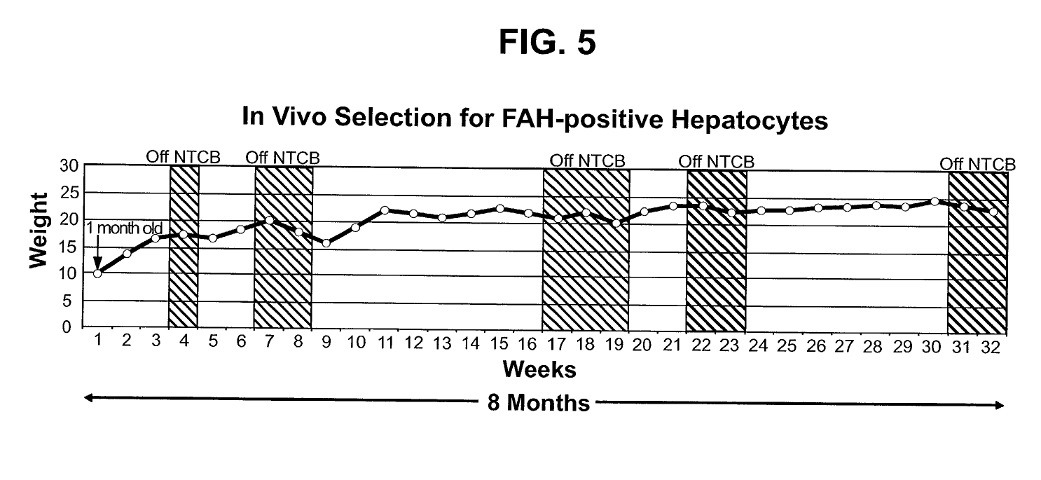

These experiments address the question of whether prior irradiation affects hepatocyte engraftment. One possibility could be that marrow ablation resulting from irradiation provides an environment permissive for the expression of HSC plasticity. We asked if HSC could give rise to hepatocytes in the absence of marrow ablation. To engraft HSC without irradiation and create a chimeric hematopoietic system, we injected (intracardiac) immunodeficient (RAG/FAH) newborn mice with wild type bone marrow cells (FIG. 4). Two months after cell injection, nucleated blood cells of the experimental animals were tested for multilineage hematopoietic engraftment. Most of the animals were engrafted at 2 months. During the next 6 to 8 months, positive selection of the engrafted cells was applied to the FAH^{-/-} mutant liver by removing NTBC from the drinking water and restarting the drug when total body weight decreased by more than 30% (see FIG. 5). Surviving mice were sacrificed after five rounds of selection (8 months after HSC transplantation) and the livers were analyzed for HSC-derived hepatocytes. A large number of hepatic nodules contained FAH-positive hepatocytes. Interestingly, HSC-derived hepatocytes were found around blood vessels, and were indistinguishable from primary adult hepatocyte-derived nodules.

Methods:

Bone marrow cells in 50-200 μl were directly injected into the heart with a ½ cc insulin syringe and 28½-gauge needle. Upon removing the needle, pressure was applied to the injection site to prevent bleeding and cell leakage.

What is claimed is:

1. A method for providing functional hepatocytes to a host animal, the method comprising:
introducing into said host animal a cell population comprising hematopoietic stem cells, wherein said hematopoietic stem cells give rise to repopulating, functional hepatocytes.

2. The method of claim 1, wherein said hematopoietic stem cells are characterized as Thy-1⁺.

3. The method of claim 2, wherein said hematopoietic stem cells are further characterized as lin^{neg}.

4. The method of claim 3, wherein said cell population is at least about 50% hematopoietic stem cells.

5. The method of claim 3, wherein said cell population is at least about 75% hematopoietic stem cells.

6. The method of claim 1, wherein said hematopoietic stem cells are mouse cells.

7. The method of claim 6, wherein said stem cells are c-kit⁺.

8. The method of claim 6, wherein said stem cells are sca-1⁺.

9. The method of claim 1, wherein said hematopoietic stem cells are human cells.

10. The method of claim 9, wherein said stem cells are CD34⁺.

11. The method of claim 9, wherein said stem cells are AC133⁺.

12. An in vitro cell culture, comprising functional regenerating hepatocytes generated from a cell population comprising human hematopoietic stem cells.

13. The in vitro cell culture of claim 12, wherein said hematopoietic stem cells are characterized as Thy-1⁺.

14. The in vitro cell culture of claim 12, wherein said hematopoietic stem cells are further characterized as lin^{neg}.

15. The in vitro cell culture of claim 12, wherein said hematopoietic stem cells are mouse cells.

16. The in vitro culture of claim 12, wherein said hematopoietic stem cells are human cells.

17. A chimeric FAH^{-/-} mouse, comprising:
functional regenerating hepatocytes generated from a cell population comprising human hematopoietic stem cells.

18. The chimeric mouse of claim 17, wherein said stem cells are CD34⁺.

19. The chimeric mouse of claim 17, wherein said stem cells are AC133⁺.

20. The chimeric mouse of claim 17, wherein said mouse is irradiated prior to introduction of said human hematopoietic stem cells.

21. The chimeric mouse of claim 17, wherein said mouse is not irradiated prior to introduction of said human hematopoietic stem cells.

22. A method of screening for genetic sequences specifically expressed in hematopoietic stem cells cultured under hepatocyte generating conditions, the method comprising:
isolating RNA from an in vitro cell culture according to claim 12 or a chimeric mouse according to claim 17
generating a probe from said RNA,
screening a population of nucleic acids for hybridization to said probe.

23. The method of claim 22, further comprising a comparison of the hybridization obtained between said hematopoietic stem cells cultured under hepatocyte generating conditions, and a differentiated cell population.

24. The method of claim 22, wherein said population of nucleic acids is represented in an array.

25. A method of screening for agents that affect the growth or differentiation of hematopoietic stem cells cultured under hepatocyte generating conditions, the method comprising:
contacting the in vitro culture of claim 12 or the chimeric mouse of claim 17 with a candidate agent, and
determining the effect of said agent on the viability, growth, or differentiation of said hematopoietic stem cells.

26. The method according to claim 25, wherein said agent is a drug suspected of toxicity on human hepatocytes.

27. The method according to claim 25, wherein said agent is a human hepatitis virus.

28. The method according to claim 25, wherein said agent is a human hepatitis virus vaccine.

29. The method according to claim 27, wherein said agent is an anti-viral agent.
